(19) United States
(12) Patent Application Publication
(10) Pub. No.: US 2026/0157930 A1
(43) Pub. Date: Jun. 11, 2026

(54) SPA CHAIR WITH A FACIAL DOME AND RELATED METHODS

(71) Applicants: Christopher Luong, Westminster, CA (US); Nguyen Le, Long Hau (VN); Nam Le, Long Hau (VN)

(72) Inventors: Christopher Luong, Westminster, CA (US); Nguyen Le, Long Hau (VN); Nam Le, Long Hau (VN)

(21) Appl. No.: 18/976,576

(22) Filed: Dec. 11, 2024

(51) Int. Cl. A61H 35/00; A61H 33/06; A61H 33/12; A61H 33/14; A61N 5/06

(52) U.S. Cl. CPC A61H 35/008; A61H 33/12; A61H 33/14; A61N 5/0616; A61H 2033/068; A61H 2033/143; A61H 2201/0149; A61H 2201/10; A61H 2201/1604; A61H 2201/5082; A61H 2203/0437; A61N 2005/0643; A61N 2005/0652

(57) ABSTRACT

A pedicure chair assembly including a chair body, a seat having a seating surface, and a facial dome connected to the chair body. The facial dome is configured to direct steam and oxygen toward a head of a user.

### FIELD OF ART

The present disclosure is generally directed to apparatuses and methods for a spa chair assembly and more particularly to a facial dome thereof for providing facial therapies and related methods.

### BACKGROUND

Facial therapy can include positioning a patient in a chair and subsequently cleansing, moisturizing, and exfoliating and massaging the patient's face. A facial therapy may also involve applying ultraviolet light to the patient's face for treating wrinkles, acne, or other dermatological issues. Pedicure therapy can include placing a patient's foot within a basin of liquid, such as moving warm or heated water within a hot tub or a jacuzzi. Some pedicure therapists seek to replicate this therapy by placing a basin in front of a chair filled with water. However, such pedicure chairs may be expensive to build and maintain, as they have more moving parts with moving liquids within them, in comparison to standard chairs. Furthermore, such facial and pedicure therapies typically require one or more technicians or therapists to apply the therapy to the patient. Thereby, facial and pedicure therapies may be expensive and cost prohibitive for some individuals.

Thus, there is a need for improved spa chairs to provide facial and pedicure therapies to a seated patient.

### SUMMARY

Broadly speaking, aspects of the invention are directed to systems and methods for providing facial therapies or treatments to a patient seated within a spa chair.

Aspects of the invention include a spa chair comprising a chair body, a seat having a seating surface, and a facial dome connected to the chair body. The facial dome is configured to direct steam and oxygen toward a head of a user.

In one embodiment, the facial dome comprises a dispenser configured to dispense the steam and the oxygen toward the head of the user.

In one embodiment, the dispenser is fitted within a dispenser cutout of a body of the facial dome.

In one embodiment, the dispenser comprises an outer surface which is flush with an outer surface of the body of the facial dome when the dispenser is seated in the dispenser cutout and an inner surface opposite the outer surface. The inner surface is directed toward the user and is flush with an inner surface of the body of the facial dome when the dispenser is seated in the dispenser cutout. The dispenser further comprises a perimeter wall extending in between the outer and inner surfaces. The perimeter wall comprises integrated mating features configured to mate with complimentary mating features of the body of the facial dome when the dispenser is seated in the dispenser cutout.

In one embodiment, the dispenser comprises a housing comprising a base, a perimeter wall extending outwardly from the base, and an open top. The housing defines an tube compartment configured to receive and house a steam tube for transporting the steam and an oxygen tube for transporting the oxygen. The dispenser further comprises a cover plate connected to and covering the housing. The cover plate comprises a steam outlet corresponding to the steam tube and an oxygen outlet corresponding to the oxygen tube.

In one embodiment, the base of the housing of the dispenser comprises a curvature which is complimentary to a curved outer surface of the facial dome such that an outer surface of the base is flush with the curved outer surface of the facial dome.

In one embodiment, the cover plate comprises a curvature which is complimentary to a curved inner surface of the facial dome such that an inner surface of the cover plate is flush with the inner surface of the facial dome, and the cover plate faces the head of the user when the cover plate is connected to the housing.

In one embodiment, the cover plate is transparent, and the cover plate is configured to cover a light source and further allow light emanating from the light source to pass therethrough such that the light shines onto the user through the cover plate.

In one embodiment, the spa chair further includes a therapy light connected to the facial dome and configured to administer a light therapy to the user.

In one embodiment, the spa chair further includes a therapy light housed within the dispenser, wherein the therapy light comprises an array of lights on a light board.

In one embodiment, the facial dome comprises a tube channel configured to receive and house a steam tube an oxygen tube therein.

In one embodiment, the facial dome is movable in between a retracted position when not in use and an extended, operational position wherein the facial dome substantially surrounds the head of the user.

In one embodiment, the spa chair further includes a steam generator configured to generate steam and provide the steam to the facial dome, and an oxygen generator configured to generate oxygen and provide the oxygen to the facial dome.

In one embodiment, the spa chair further includes a system controller operably coupled to the steam generator, the oxygen generator, and a temperature sensor located on or next to the facial dome. The temperature sensor is configured to sense a temperature of air within the facial dome and further detect a threshold temperature of the air within the facial dome. The system controller is configured to activate the steam generator and/or the oxygen generator upon an input command. The system controller is configured to deactivate the steam generator upon receiving a threshold temperature signal from the temperature sensor when the temperature sensor detects the threshold temperature.

Aspects of the invention include a pedicure chair assembly comprising a chair body, a seat having a seating surface, and a basin located elevation-wise below the seating surface. The basin comprises walls and an open top. The basin defines an open cavity for holding water therein. The pedicure chair assembly further comprises a mist generator configured to generate and supply mist to the basin such that the mist obscures a view of the water within the basin.

In one embodiment, the mist generator is housed within the chair body.

In one embodiment, the pedicure chair assembly further comprises a mist dispenser spout located next to and elevation-wise above the basin. The mist dispenser spout is fluidly coupled to the mist generator by a mist tube.

In one embodiment, the mist generator comprises a water inlet, a water tank fluidly coupled to the water inlet and configured to house water therein, an ultrasonic mist maker disposed within the water tank and configured to generate mist from the water within the water tank, an ultraviolet (UV) light disposed at a top of the water tank and configured to disinfect the water and/or mist, and a mist outlet fluidly coupled to the water tank.

In one embodiment, the mist generator further comprises a fan configured to blow mist out through the mist outlet and into the basin.

Aspects of the invention include a pedicure chair assembly comprising a chair body, a seat having a seating surface, and a basin located elevation-wise below the seating surface. The basin comprises walls and an open top. The basin defines an open cavity for holding water therein. The pedicure chair assembly further comprises a steam generator disposed within the chair body and configured to generate steam, an oxygen generator disposed within the chair body and configured to generate oxygen, and a mist generator disposed within the chair body and configured to generate and supply mist to the basin such that the mist obscures a view of the water within the basin. The pedicure chair assembly further comprises a facial dome connected to the chair body. The facial dome is fluidly connected to the steam generator and the steam generator. The facial dome is configured to respectively receive the steam and the oxygen from the steam generator and the oxygen generator, respectively, and direct the steam and the oxygen toward a head of a user.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 illustrates a perspective view of a spa chair with a mist generator, a steam generator, an oxygen generator, and a facial dome for providing facial therapies or treatments to a user seated in the spa chair.

FIG. 2 illustrates a schematic diagram of the system controller of the spa chair.

FIG. 3 illustrates a partial schematic and perspective view of the mist generator, the steam generator, the oxygen generator, and the facial dome of the spa chair.

FIG. 4 illustrates a perspective view of the facial dome of FIG. 1, which includes a body, steam and oxygen tubes, and a dispenser connected to the body. The dispenser is illustrated in a partial cutaway view to better show the steam and oxygen tubes housed therein.

FIGS. 5A-5B illustrate perspective views of the dispenser of the facial dome of FIG. 1. FIG. 5A illustrates an exploded view of the dispenser, which generally includes a housing, a light source, and a cover plate. FIG. 5B illustrates a perspective view of a side of the housing of FIG. 5A, showing a mating feature thereon for connecting the housing to the body of the facial dome.

FIG. 6 illustrates a perspective view of the mist generator of the spa chair of FIG. 1.

FIG. 7 illustrates a perspective view of the steam generator of the spa chair of FIG. 1.

FIGS. 8A-8B illustrate perspective views of the oxygen generator of the spa chair of FIG. 1. FIG. 8A illustrates a perspective view of the oxygen generator which includes a compressor and a nitrogen filter. FIG. 8B illustrates a cross-sectional view of the nitrogen filter of FIG. 8B, taken across a longitudinal axis thereof.

FIG. 9 illustrates a flowchart of a method for providing one or more facial therapies or treatments to the user seated in the spa chair.

### DETAILED DESCRIPTION

The detailed description set forth below in connection with the appended drawings is intended as a description of the presently preferred embodiments of a spa chair apparatus with a facial dome, in accordance with aspects of the present devices, systems, and methods, and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

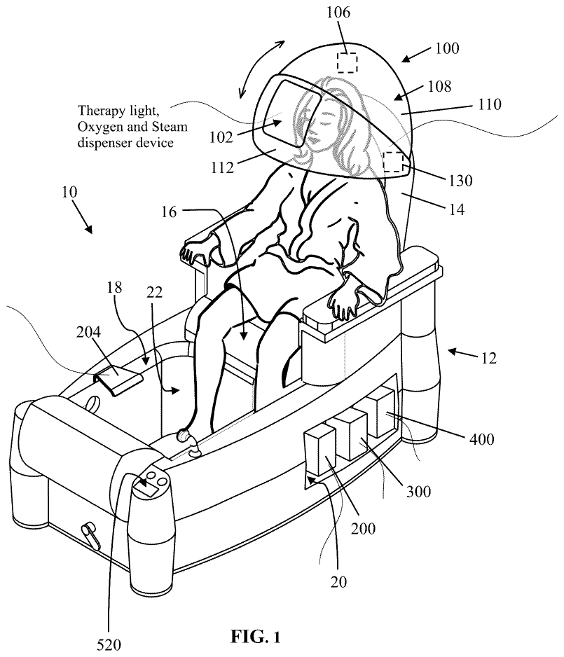

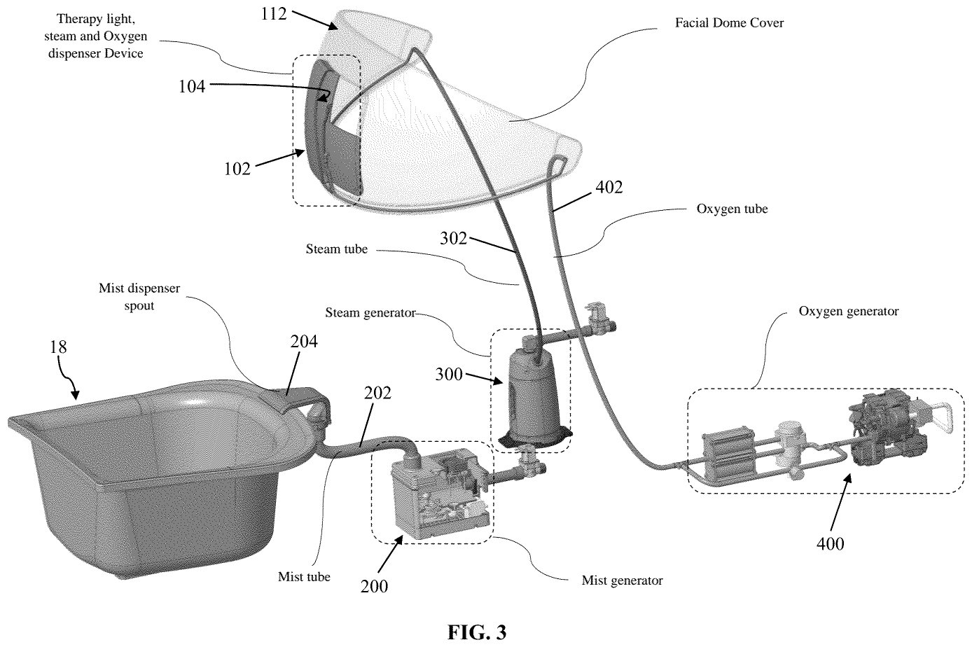

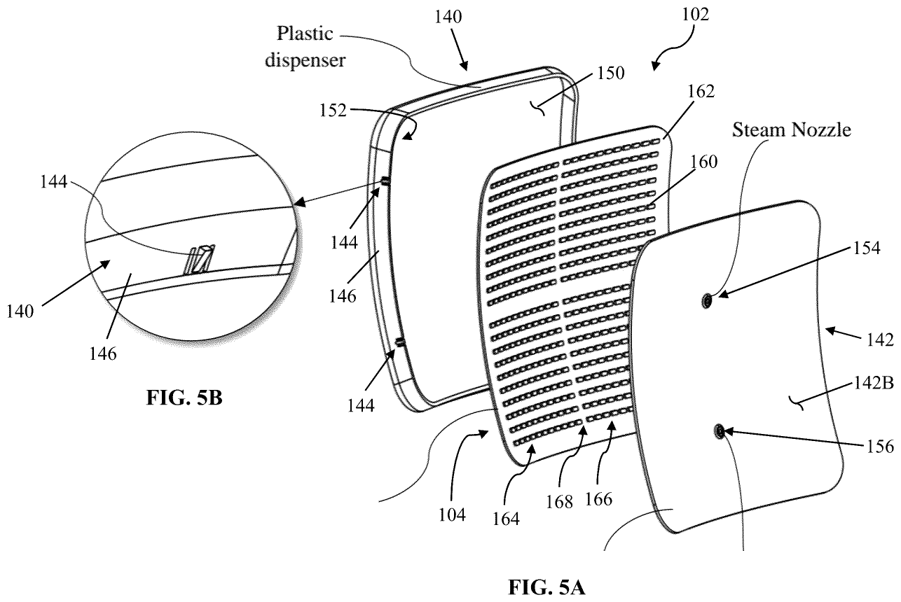

Referring initially to FIGS. 1-3, in one embodiment, a spa chair 10 may be configured as a pedicure chair assembly 10 which generally comprises a chair body 12, a seat 14 having a seating surface 16 upon which a user can sit, a basin 18 for holding water with optional pedicure therapies, and a facial dome 100 for providing one or more facial therapies or treatments (or face, head, scalp, hair, and/or neck therapies or treatments) to the user as the user is seated in the spa chair 10. The spa chair 10 may further include a mist generator 200 that may provide mist to the basin 18 via a mist tube 202 and a mist dispenser spout 204, a steam generator 300 that may provide steam to the facial dome 100 via a designated steam tube 302, and an oxygen generator 400 that may provide oxygen to the facial dome 100 via a designated oxygen tube 402 (FIG. 3). Unless the context indicates otherwise, the various tubes can be off-the-shelf tubes capable of handling the services in question and the specific label, such “oxygen” tube merely serves as a reference or a nomenclature. In one embodiment, the facial dome 100 may include a dispenser 102 with a built-in therapy light or light source 104 (FIG. 5A) for selectively dispensing the steam and oxygen whilst also providing one or more light therapies or treatments to the user, as discussed further herein.

The chair body 12 may house the generators 200, 300, 400 within a generator compartment 20 (FIG. 1) with separate compartments or locations contemplated for each of the plurality of generators. The generator compartment 20 may or may not be enclosed by an access panel or door (not shown). In one embodiment, the generator compartment 20 may be open to allow easy access to the generators 200, 300, 400. In one embodiment, the generator compartment 20 may be an internal compartment which is accessible via a panel or door, which can be transparent, opaque, or semi-opaque and can include appropriate latches or locks. In one embodiment, the chair body 12 may comprise multiple compartments 20 for respectively housing the generators 200, 300, 400, wherein each compartment has a respective panel or door to access the generator 200, 300, 400 therein. The chair body 12 may further include internal holes, channels, or passageways for accommodating the electrical and fluid lines or tubes of the various generators 200, 300, 400 of the spa chair 10. In less preferred embodiment, a module that is separate from the chair body comprising a housing is utilized to house the one or more generators 200, 300, 400. Outlets from the one or more generators located in the separate housing can connect to a header or a connection panel having corresponding connectors for distributing the outlets from the generators to the basin and the facial dome.

The basin 18 may define an open cavity for holding water and the user's feet therein, when the person is in a seated position on the seat 14. In one embodiment, the basin 18 may include a bottom, walls extending upwardly from the bottom, and an open top which allows the user to easily fit their feet within the basin 18. The basin 18 is located, elevation-wise, below the seating surface 16 of the seat 14. In one embodiment, the basin 18 may be unitarily formed with the chair body 12, wherein the chair body 12 may define the basin 18 itself. In one embodiment, the basin 18 may be a separate component, such as a separately formed basin 18, that is placed in a frame or structure (e.g., the chair body 12 itself) for supporting the separately formed basin 18 in front of the seat 14. For example, in one embodiment, the chair body 12 may comprise a cavity that is sized and disposed to accept a separately formed and complimentary basin 18. Therein, the chair body 12 may additionally comprise an attachment mechanism that holds the basin 18 in place within the cavity, for example by using clamps, fasteners, a clasp, matching indents and detents, or an elastic band.

In some embodiments, the chair body 12 and the basin 18 could comprise different materials, similar to an opening of a counter-top for a sink. In such embodiments, the chair body 12 could comprise materials that are not waterproof while the basin 18 could comprise materials that are waterproof without damaging the non-waterproof portions of chair body 12. In one embodiment, the basin 18 may comprise a waterproof material, such as thermoplastic, ceramic, resin, or glass, and could be made to be opaque, translucent, or transparent, however the basin 18 may be made of any suitable material(s). The basin may include an opening for accommodating a magnetic drive pump 22 (shown schematically) that circulates water jets within the basin to enhance the pedicure experience.

In operation, the basin 18 may be completely or partially filled with water, such as with a pitcher, a hose, or via one or more water supply outlets which are fluidly coupled to a water source. In some embodiments, the basin 18 can used in tandem with auxiliary devices, such as a footrest, a basin water steamer, one or more jet pumps 22, and/or a basin cover (not shown). Exemplary circulating jet pumps for a pedicure chair are described in U.S. Pat. Nos. 8,272,079 and 11,679,062, the contents of each of which are expressly incorporated herein by reference. An exemplary basin cover is described in U.S. Pat. No. 12,121,494, the contents of which are expressly incorporated herein by reference.

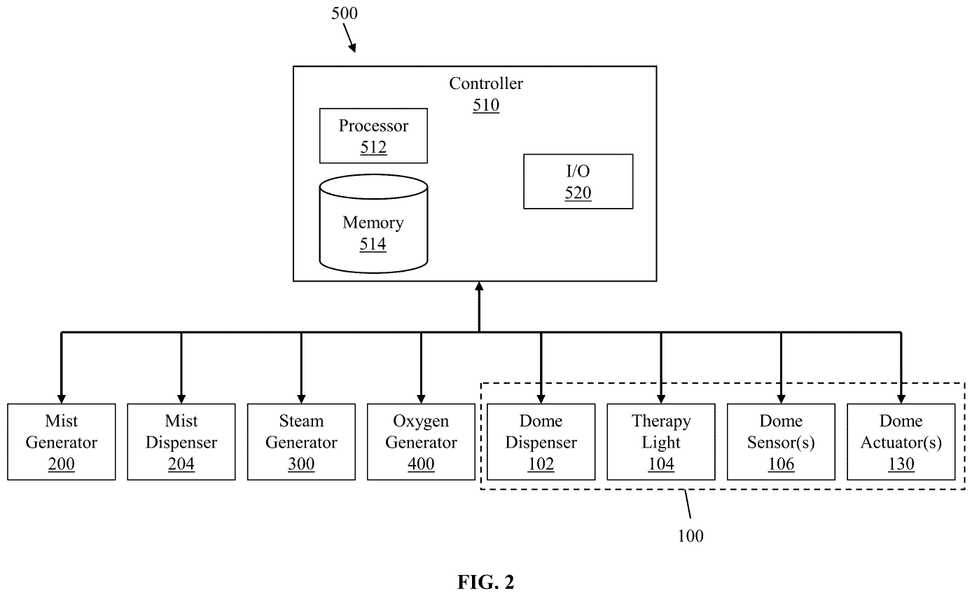

Referring to FIG. 2, in one embodiment, a spa chair control system 500 for controlling operations of the generators is shown, which may include a system controller 510 with a processor 512 and a memory 514 that may be operably connected to and control the operation(s) of one or more of the mist generator 200, the mist dispenser spout 204, the steam generator 300, the oxygen generator 400, the facial dome 100 as discussed in more detail herein, and the basin 18. The processor 512 may be configured to execute software instructions stored on a tangible, non-transitory computer readable storage medium (e.g., hard drive, solid state drive, RAM, flash, ROM, etc.). The software instructions configure the system controller 510 and server to execute the various functionality of the spa chair 10 and operational components thereof. As used herein, a “system controller” may comprise any suitable combination of computing or computer devices, such as desktops, laptops, wearable devices, cellular phones, blades, servers, interfaces, systems, databases, agents, peers, engines, modules, or controllers, operating individually or collectively. In one embodiment, the system controller 510 may include a simple computer system having a programmable EEPROM chip that saves simple instructions, for example an instruction to activate the mist, oxygen, and/or steam generators 200, 300, 400 and/or the therapy light 104, until a certain and corresponding threshold and/or time limit is detected, upon which the chip may transmit a command to deactivate the mist, oxygen, and/or steam generators 200, 300, 400 and/or therapy light 104.

In one embodiment, the control system 500 may further include an I/O device 520 (FIG. 1), such as a control panel with display or touchscreen that displays one or more selectable options and/or parameters or states of the spa chair 10, such as a measured water temperature, a desired water temperature, an elapsed time, a total time of a given therapy, a massage status, a light status, a generator status, a desired temperature within the facial dome 100, a desired amount of steam, a desired amount of oxygen, or any other desired parameter. The control panel 520 may comprise a graphical user interface (GUI) with selectable icons or other generated manipulable elements that allow the user or technician to control the various operations of the spa chair 10. In one embodiment, the control panel 520 may include physical buttons, such as switches, dial knobs, on/off switches, etc., and an emergency override button, as non-limiting examples. In alternative examples, the control panel can include soft keys that change with different screens to control different functions. In one embodiment, the spa chair 10 may include multiple control panels 520, including a user control panel located on the spa chair 10 at a first location for the user and a technician control panel located on the spa chair 10 at a second location, which respectively allow the user and the technician to respectively control the spa chair 10 as desired.

One or more sensors could be operably coupled to the system controller 510 to assist in executing the commands thereof. Therein, in some embodiments, the spa chair 10 may initiate, adjust, and/or terminate the various therapies or treatments provided to the user, in real time and responsive to data sensed by the one or more sensors. In one embodiment, the spa chair 10 may include one or more basin sensors, mist sensors, and/or dome sensors 106. In one embodiment, one or more basin sensors may sense the temperature and/or water level of the water within the basin 18. Exemplary basin sensors for a pedicure chair are described in U.S. Pat. No. 12,121,494, the contents of which are expressly incorporated herein by reference. In one embodiment, the spa chair 10 may include one or more mist sensors (not shown), e.g., optical sensors, for detecting an amount of mist within the basin 18. Other commercially available switches, control mechanisms, thermocouples, and sensors are contemplated and can be used with the spa chair 10, such as an on/off button and switches for controlling other functions incorporated with the spa chair 10, such as to control moving massage elements or for providing music.

In one embodiment, the spa chair 10 may include one or more dome sensors 106, as shown in phantom in FIG. 1. The one or more dome sensors 106 may include a temperature sensor, a gas sensor, such as an electrochemical or optical sensor for sensing an amount of oxygen within the dome, and/or a position sensor for sensing a position of the facial dome 100, as discussed further herein. In some embodiment, the spa chair 10 may not include a dome sensor 106, e.g., may omit the sensors or may have fewer than the listed sensors.

In one embodiment, the spa chair 10 may include a temperature sensor located on or near the facial dome 100 to detect the temperature of the air (or the user) within the facial dome 100. The temperature sensor may be configured to send a threshold temperature signal, i.e., a maximum temperature limit signal, to the system controller 510 upon sensing a threshold temperature within the facial dome 100, such as for example 80 degrees Fahrenheit (26.67 degrees Celsius), plus or minus 15 degrees. The threshold temperature may be a predetermined temperature stored in the memory and/or selectable by the user or technician via the control panel 520. Responsive to receiving the threshold temperature signal, the system controller 510 may cool the facial dome 100 by ceasing to provide steam to the facial dome 100 and/or increasing the airflow provided into the facial dome (by increasing the amount of airflow from the steam generator 300, without steam therein, and/or the oxygen generator 400).

In one embodiment, the spa chair 10 may include a gas sensor configured to detect an amount of oxygen within the facial dome 100. The gas sensor may be configured to send a cutoff, threshold oxygen signal to the system controller 510 upon sensing a threshold oxygen level within the facial dome 100. Responsive to receiving the threshold oxygen signal, the system controller 510 may deactivate the oxygen generator 400. As yet another example, in one embodiment, the spa chair 10 may include a light sensor to detect an amount and/or position of light within the facial dome 100. For instance, the presence of steam within the facial dome 100 may impact the dispersal and strength of the light within the facial dome 100, and the system controller 510 may manipulate the therapy light to accordingly compensate for a given ratio of steam, oxygen, and ambient air within the facial dome 100.

In one embodiment, the spa chair 10 may include a dome position sensor that detects a position of the facial dome 100. In particular, the dome position sensor may detect whether the facial dome 100 is in its stowed or operational position. The position sensor may send a position signal to the system controller 510, corresponding to the position of the facial dome 100, and thereafter the system controller 510 may turn on or off the steam generator 300, the oxygen generator 400, and/or the therapy light depending upon the received position signal.

In one embodiment, the facial dome 100 may not include any dome sensors 106 for controlling the various operations of the facial dome 100. Instead, the system controller 510 may (de)activate the steam generator 300 and oxygen generator 400 based on user input and/or a timer. For example, in one embodiment, the system controller 510 may automatically turn off the steam generator 300, the oxygen generator 400, and/or the therapy light 104 after a predetermined time limit therefor. In one embodiment, the system controller 510 may automatically control the steam generator 300, the oxygen generator 400, and/or the therapy light according to a predetermined sequence, cycle, duration, amount, etc., to provide a desired therapeutic sequence of steam, oxygen, and/or light.

In one embodiment, the spa chair 10 may include a user sensor for detecting the position and/or various characteristics, traits, e.g., face position and size thereof, or statuses of the user, e.g., the user's temperature and treatment elapsed time. For example, in one embodiment, the user sensor may comprise a pressure sensor, an optical or infrared sensor, and/or a temperature sensor for monitoring the user. The user sensor may be configured for providing the sensed data to the system controller 510, and upon receiving the sensed data the system controller 510 may process the data to determine a current state and/or position of the user and thereafter alter the generation of steam, oxygen, and/or application of the therapy light.

In one embodiment, the mist dispenser spout 204 may be movable and/or selectively closeable by the system controller 510, based upon sensed data from a mist sensor and/or an input command. For example, in one embodiment, the mist dispenser spout 204 may be rotatably mounted to the chair body 12 via an actuator, such as a servo motor. Therein, the system controller 510 may rotate the mist dispenser spout 204 to direct mist toward the front or rear of the basin 18. Additionally, in one embodiment, the mist dispenser spout 204 may include an adjustable and/or closeable baffle or diverter at its distal end for redirecting the mist as desired. The diverter may be movable by an actuator, such as a servo motor, which is operably connected to the system controller 510. In another example, an inline control valve may be used to shut off the flow of mist or to modulate the flow of the mist, such as to throttle the flow based on a sensed data. In some embodiments, the mist dispenser spout 204 and/or diverter thereof may be manually adjustable by the user or technician.

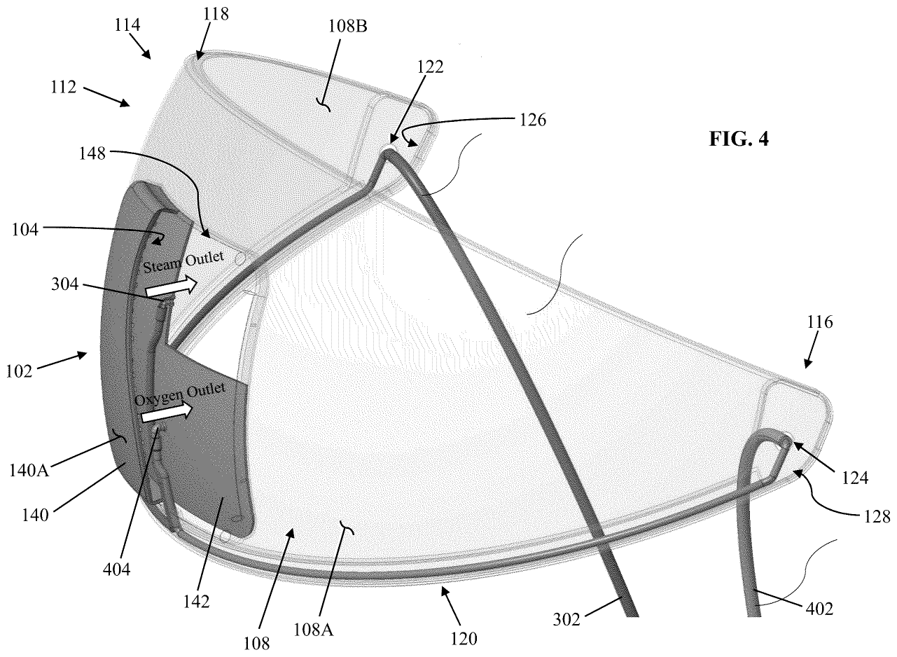

Referring to FIGS. 4 and 5A-5B, in one embodiment, the facial dome 100 in accordance with aspects of the invention may comprise a dispenser 102 configured for dispensing steam and/or oxygen and/or emitting light from the therapy light 104 when providing one or more facial therapies or treatments to the user. FIG. 4 illustrates a front perspective view of the facial dome 100, wherein the facial dome 100 is shown in skeleton lines and the dispenser 102 is partially cutaway to better illustrate the steam and oxygen tubes 302, 402 that are routed through parts of the cove 112 and the dispenser 102.

The facial dome 100 may be configured as a single part with a monolithic body 108 or a multipart body 108 with two or more components. In one embodiment, the body 108 of the facial dome 100 may comprise a facial dome base 110 and a facial dome cover 112 movably connected to the facial dome base 110 (FIG. 1). For instance, the facial dome cover 112 may be pivotally or slidably connected to the facial dome base 110 via one or more bolts, pivot pins, bosses and recesses, tongue and grooves, tracks, etc. In one embodiment, the facial dome cover 112 may comprise an arcuate body that is complimentary to the shape and size to the facial dome base 110. The facial dome cover 112 may be slightly larger or smaller to fit over and on top of or within and underneath the facial dome base 110, respectively. In one embodiment, as shown in FIG. 1, the facial dome cover 112 may have an inner surface profile with a radius that is slightly larger than a radius of an outer surface profile of the facial dome base 110, allowing the facial dome cover 112 to fit over and rotate above and onto the facial dome base 110. The facial dome base 110 may form approximately three-quarters of a dome, and the facial dome cover 112 may form approximately one-quarter of the dome, wherein together the facial dome base 110 and cover 112 form a complete dome. In one embodiment, the facial dome cover 112 may include a widened section 114 and tapered, narrower ends 116, forming a wedge-like shell bounded by an upper rim 118 and a lower rim 120 (FIG. 4). The facial dome 100 may comprise any desired material, such as a polymer, a glass, a metal, and/or a wooden material. In one embodiment, the facial dome 100 may be comprised of a clear plastic material. In some examples, the facial dome base 110 and/or the facial dome cover 112 may include adjustable louvers to enable the user or the technician to

In one embodiment, the facial dome cover 112 may comprise one or more tube receiving holes, one or more channels, compartments, and/or holes for receiving and internally housing the steam and oxygen tubes 302, 402, electrical wires (not shown), or other cables, therein for supplying steam and oxygen to the dispenser 102 and electrical power to the therapy light 104. For example, in one embodiment as shown in FIG. 4, the facial dome cover 112 may include a pair of tube holes 122, 124 at each tapered end 116 of the facial dome cover 112, that open into one or more internal tube channels or compartments 126, 128 which may lead to the dispenser 102 (FIG. 4). Each tube channel 126, 128 may extend from each tapered end 116 to a forwardmost (or outermost) point located underneath the dispenser 102, following the arcuate profile of the facial dome cover 112. In one embodiment, the one or more tube channels 126, 128 may be located next to the lower rim 120 of the facial dome cover 112, keeping the weight of the steam and oxygen tubes 302, 402 underneath a center of mass of the facial dome cover 112. In one embodiment, the steam tube 302 and the oxygen tube 402 may respectively extend from the steam and oxygen generators 300, 400, through the chair body 12 (e.g., in designated tube channels), into a respective left-side and right-side tube hole 122, 124 in the facial dome 100, through the internal tube channel(s) 126, 128, and into the dispenser 102 (e.g., via corresponding holes in the top of the tube channel(s) 126, 128 and the bottom of the dispenser 102).

In one embodiment, the facial dome 100 may be configured to manually and/or automatically move in between a retracted, stowed position (not shown) and an extended, operational position (as shown in FIG. 1). In the stowed position, the facial dome 100 may reside at the top and/or rear of the seat 14. Thereby, the facial dome 100 may not extend around the head of the user in the stowed position. In the operational position (FIG. 1), the facial dome 100 may be expanded or rotated forwardly of the seat 14. Thereby, the facial dome 100 may substantially surround and cover the head of the user in the operational position.

In one embodiment, the facial dome 100 may be manually movable by the user or technician. In one embodiment, the facial dome 100 may further comprise a dome actuator 130 (shown in phantom in FIG. 1) that automatically moves the facial dome 100 in between its stowed and operational positions. The dome actuator 130 may comprise one or more electric motors, mechanical arms or levers, pistons, etc., that may rotate or otherwise move the facial dome 100, or a portion of the body 108 thereof, upon receiving an input command. The dome actuator 130 may be mounted to the chair and/or a portion of the body 108 of the facial dome 100. In one embodiment, wherein the facial dome 100 comprises a multipart body 108 with an automatic facial dome cover 112, a pair of actuators 130 may rotatably mount the facial dome cover 112 to the facial dome base 110. Thereby, upon receiving an input command, the system controller 510 may automatically raise or lower the facial dome cover 112 into its retracted or operational positions, respectively.

FIG. 5A illustrates an exploded view of an embodiment of a dome dispenser 102 that is usable with the facial dome 100 in accordance with aspects of the invention. The dispenser 102 may include a housing 140, a cover plate 142 removably connected to the housing 140, and an enclosed therapy light 104. FIG. 5B is an enlarged sectional view of the base of FIG. 5A and illustrates an integrated mating feature 144 on a side wall 146 of the housing 140, as discussed in more detail below. In one embodiment, the facial dome 100 may comprise a dispenser cutout 148 (FIG. 4) that is sized and shaped to receive the dome dispenser 102 therein. The size and shape of the dispenser cutout 148 may be complimentary to the size and shape of the dome dispenser 102. In one embodiment, wherein the facial dome 100 includes a multipart body 108, the facial dome cover 112 may include a dispenser cutout 148 for mounting the dispenser 102 therein. In some embodiments, the facial dome 100 may include an integrated dispenser and thus may not include a dispenser cutout 148. In such an embodiment with an integrated dispenser, the facial dome may include a removable cover plate that covers a recess or compartment on the inside surface of the facial dome 100. The dispenser 102 may comprise any desired material, such as a polymer. In one embodiment, the dispenser may be comprised of a clear plastic material.

In one embodiment, the housing 140 of the dispenser 102 may comprise a base 150, a perimeter wall 146 (defining the sidewalls 146 of the housing 140) that circumferentially surrounds and extends outwardly from the base 150, and an open top end 152. The housing 140 defines a tube compartment which is selectively coverable by the cover plate 142. The perimeter wall 146 of the housing 140 may comprise the integrated mating features 144 that are configured to mate with complimentary mating features of the body 108 of the facial dome 100 when the dispenser 102 is seated in the dispenser cutout 148. For example, in one embodiment as shown in FIG. 5B, the perimeter wall 146 of the housing 140 may include a plurality of snap-fit tabs 144 that are received within corresponding recesses (not shown) in the periphery of the dispenser cutout 148 of the body 108 of the facial dome 100. However, in some embodiments, the mating features 144 between the housing and the facial dome 100 may include fasteners, latches, tongue and groove mating features, protrusions and recesses, etc. To accommodate the steam and oxygen tubes 302, 402, the bottom section of the perimeter wall 146 may include a pair of tube holes or cutouts (not shown) that receive the steam and oxygen tubes 302, 402 therethrough, and the tube compartment 140 defined by the housing 140 may be configured to receive and house the distal ends of the steam and oxygen tubes 302, 402.

The cover plate 142 of the dispenser 102 may include a steam outlet 154 and an oxygen outlet 156 for respectively receiving the terminal ends 304, 404 of the steam and oxygen tubes 302, 402, allowing the steam and oxygen to be dispensed therefrom (FIG. 4). The steam outlets can embody openings formed through the cover plate. In some examples, an air distribution eye-ball type nozzle can be positioned at each outlet to directionally control the flow stream. The steam and oxygen outlets 154, 156 may be located adjacent to one another, allowing the steam and oxygen to mix with one another upon exiting the cover plate 142. Thereby, in one embodiment, the cover plate 142 may be configured to mix the steam and the oxygen before the steam and oxygen contact the user. A static mixer, which comprises helix baffles, may be used to facilitate mixing. In one embodiment, the cover plate 142 may be removably connected to the housing 140. For example, in one embodiment, the cover plate 142 may be snap-fitted onto the housing 140. Hence, the outer periphery of the cover plate 142 may substantially match the inner periphery of the perimeter wall 146, allowing the cover plate 142 to be compression fit against the perimeter wall 146 of the housing 140. In some embodiments, the cover plate 142 may be connected to the housing 140 via fasteners, corresponding protrusions and recesses, or other integrated mating features. The cover plate may comprise any desired material, such as plastic. In one embodiment, the cover plate 142 may comprise a transparent material. Thereby, the cover plate 142 may be configured to cover and protect the therapy light 104 whilst also allowing the light emanating from the light source to pass therethrough such that the light shines onto the user. Thereby, the material of the cover plate 142 and the position of the outlets 154, 156 therein, and the resulting position of the steam and oxygen tubes 302, 402, may not obstruct the operation of the therapy light 104. In other words, since the steam and oxygen outlets 154, 156 are vertically aligned with one another, the steam and oxygen tubes 302, 402 may become grouped together, minimizing a profile of the steam and oxygen tubes 302, 402 such that the therapy light 104 is unobstructed by the steam and oxygen tubes 302, 402.

In one embodiment, the therapy light 104 may be housed within the housing 140 of the dispenser 102. More particularly, the therapy light 104 may be located between the inside surface of the base 150 of the housing 140 and the steam and oxygen tubes 302, 402. In one embodiment, as shown in FIG. 4, the therapy light 104 may directly contact the inside surface of the base 150 of the housing 140 and be fully encircled by the perimeter wall 146 of the housing 140. The therapy light 104 may comprise a single light or multiple lights. The therapy light 104 may emit visible light, infrared light, and/or ultra violet (UV) light. In one embodiment, the therapy light 104 may comprise an array of lights 160 on a light board 162 (FIG. 4). In one embodiment, the therapy light 104 may comprise an array of light emitting diodes (LEDs) 160 on an LED board 162. The LED lights 160 may be configured to emit any color in the visible light spectrum. In one embodiment, the light board 162 may be complimentary to the size and shape of the perimeter wall 146 of the housing 140. The lights 160 on the light board 162 may be arranged in juxtaposed left and right banks or columns 164, 166. The left and right banks 164, 166 may differ from one another such that the left bank 164 includes fewer lights than the right bank 166. The left and right banks 164, 166 of the therapy light 104 may be separated by a gap or empty space 168 of the light board 162. The gap 168 may not include any lights 160 therein. The gap 168 between the left and right banks 164, 166 of lights 160 may accommodate the steam and oxygen tubes 302, 402, allowing the left and right banks 164, 166 to emit light without obstruction from the steam and oxygen tubes 302, 402. Thereby, in one embodiment, the steam and oxygen outlets 154, 156 may be vertically aligned and coplanar with the gap 168 of the light board 162. In other words, a vertical axis extending in between the outlets 154, 156 may be aligned with a longitudinal axis of the gap 168 of the light board 162. Thereby, when assembled, the steam and oxygen tubes 302, 402 may be substantially aligned with the gap 168 in between the lights 160 on the light board 162 and thus do not substantially block light emanating from the therapy light 104. In one embodiment, the left and right banks 164, 166 may comprise differing lights 160, wherein one bank includes lights which emit visible light and the other bank includes ultraviolet lights. In one embodiment, the facial dome 100 may include additional therapy lights 104 disposed at the top, bottom circumference, front, rear, and/or sides of the inner surface of the facial dome 100. Thereby, the therapy light(s) 104 may apply one or more light therapies anywhere (e.g., a full 360 degrees) around the head and/or face of the user.

In one embodiment, the dispenser 102 may be complimentary to the size and shape of the dispenser cutout 148 in the body 108 of the facial dome 100. For example, the dispenser 102 may have a substantially rectangular cross-section, with rounded or beveled corners, and the dispenser cutout 148 may have a complimentary rectangular cross-section which receives the dispenser 102 therein. However, the dispenser 102 and the dispenser cutout 148 of the facial dome 100 may each have any desired and complimentary shape and size.

In one embodiment, the curvature of the dispenser 102 may correspond to the curvature of the body 108 of the facial dome 100 such that the dispenser 102 is flush with the facial dome 100 when mounted thereto, creating an apparent smooth transition between the dispenser 102 and the body 108 of the facial dome 100. For example, in one embodiment, an outer surface 150A of the base 150 of the housing 140 of the dispenser 102 (which faces outwardly away from the seat 14 and user seated therein) may be flush with a curved outer surface 108A of the body 108 of the facial dome 100 when the dispenser 102 is seated in the dispenser cutout 148 (FIG. 4). An inner surface 142B of the cover plate 142 (opposite the outer surface 140A of the housing 140 and which faces inwardly toward the seat 14 and the user seated therein) may be flush with a curved inner surface 108B of the body 108 of the facial dome 100 when the cover plate 142 is connected to the housing 140 of the dispenser 102, when the dispenser 102 is seated in the dispenser cutout 148 (FIG. 4). The perimeter wall 146 of the housing 140 of the dispenser 102 may have a depth that matches a depth of the dispenser cutout 148 in the facial dome 100.

In one embodiment, the facial dome 100 may include a plurality of dispensers 102. For example, in one embodiment, four dispensers 102 may be equidistantly spaced about the inner periphery of the facial dome 100. The plurality of dispensers 102 may be located on the facial dome base 110 and/or the facial dome cover 112. The plurality of dispensers 102 may be substantially the same or each dispenser 102 may differ from one another in design and function. For example, the facial dome 100 may include separate steam dispensers for dispensing steam, oxygen dispensers for dispensing oxygen, and therapy lights spaced about the inside of the facial dome 100.

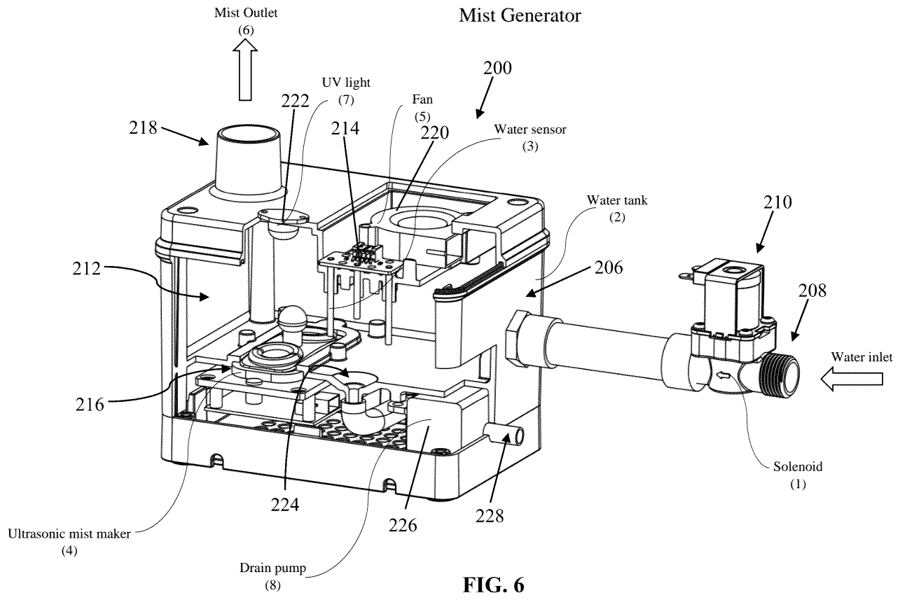

Referring to FIG. 6, there is shown a perspective and partial cutaway view of an exemplary embodiment of a mist generator 200 in accordance with aspects of the invention. The mist generator 200 may be housed within the chair body 12, such as in the generator compartment. In one embodiment, the mist generator 200 may include a housing 206, a water inlet 208 connected to a water source, and a solenoid valve 210 for selectively intaking water into a water tank 212. A water sensor 214 may be located within the water tank 212 for sensing a water level within the water tank 212. The water sensor 214 may be any desired commercially available water sensor, such as an optical sensor, a probe sensor, etc. A mist maker 216 may be located within the water tank 212, underneath a water line. For example, in one embodiment, the mist maker 216 may be located at the bottom of the water tank 212. The mist maker 216 may comprise any desired piezoelectric transducer for oscillating the water via ultrasonic frequency vibration. The mist maker 216 may generate a fine and dry fog which evaporates into the air. The fog may then travel out through a mist outlet 218 at the top of the water tank 212, and thereafter travel through the mist tube 202 and exit through the mist dispenser spout 204 into the basin 18 (FIG. 3). The mist may then accumulate within the basin 18, covering and obscuring the water within the basin 18. Thereby, the mist may create a more aesthetically pleasing appearance by covering or otherwise hiding the user's feet within the basin 18. Optionally, a fan 220 may be included near the top of the water tank 212 to forcibly blow the mist out through the mist outlet 218 and into the basin 18. The fan 220, in one embodiment, may comprise a rotary fan or a blower. A sterilizer and/or water filter 222 may be housed within the water tank 212 to purify the generated mist and/or the water within the water tank 212. For example, in one embodiment, an ultraviolet (UV) light 222 may be located at a top of the water tank 212 for sterilizing the mist before the mist exits through the mist outlet 218. To empty the water tank 212, a drain outlet 224 may be located at the bottom of the water tank 212. The water may drain via gravity or via a drain pump 226. For example, in one embodiment, a drain pump 226 may forcibly pump the water out of the water tank 212. The water from the water tank 212 may exit through a water outlet 228 near the bottom of the housing 206. If equipped, the drain pump 226 may comprise any desired commercially available drain pump.

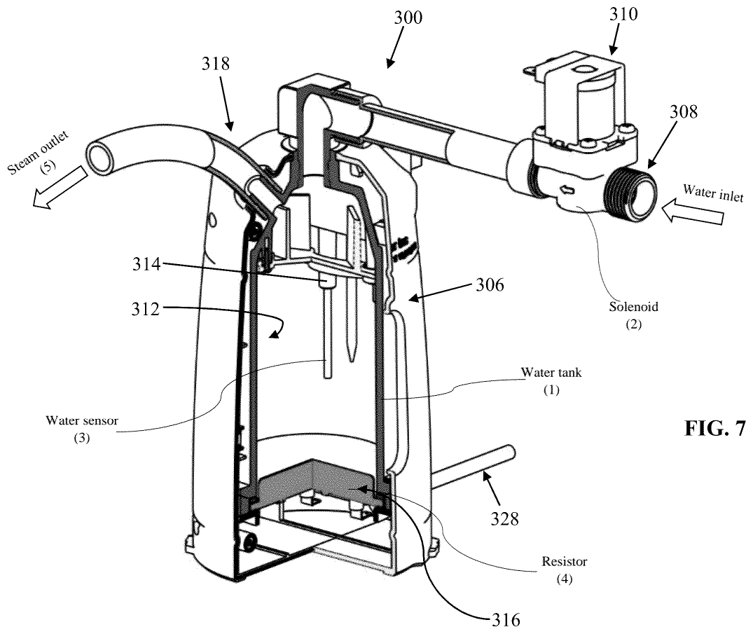

Referring to FIG. 7, there is shown a perspective and partial cutaway view of an exemplary embodiment of a steam generator 300. The steam generator 300 may be housed within the chair body 12, such as in the generator compartment. In one embodiment, the steam generator 300 may include a housing 306, a water inlet 308 connected to a water source, and a solenoid valve 310 downstream of the water inlet 308 for selectively intaking water into a water tank 312 there below. A water sensor 314 may be located within the water tank 312 for sensing a water level within the water tank 312. The water sensor 314 may be any desired commercially available water sensor, such as an optical sensor, a probe sensor, etc. The solenoid valve 310 may allow water into the water tank 312 until the water sensor 314 senses a sufficient amount of water has filled up the water tank 312, at which point the system controller 510 may close the solenoid valve 310 upon receiving a desired fill-level signal from the water sensor 314. Thereafter, the system controller 510 may begin the steam generation process. To generate the steam, an electric heating element 316 may be disposed near the bottom of the water tank 312 for heating the water therein. To generate the steam, the heating element 316 may boil or nearly boil the water, e.g., heating the water to approximately 212 degrees Fahrenheit (100 degrees Celsius). In one embodiment, the heating element 316 may include a stainless steel plate and a nichrome resistor underneath the plate. However, the steam generator 300 may include any desired heating element. As the water boils, steam may begin to rise and exit through a steam outlet 318 at the top of the water tank 312. The steam may travel through the steam tube 302 and exit through its terminal end 304 at the steam outlet 154 of the dispenser 102 of the facial dome 100 (FIGS. 3 and 5A).

In one embodiment, a dome sensor 106 in the form of a steam sensor, located on or near the facial dome 100, may sense and provide a stop and start signal to the system controller 510 to continually and/or intermittently provide a desired amount of steam to the facial dome 100. In one embodiment, a steam timer may monitor the duration at which the water is boiling, and the system controller 510 may turn on or off the steam generator 300 at preset time intervals. Therein, the amount of steam generated by the heating element 316 may be indirectly sensed by the timer and the water sensor 314 detecting whether the water is boiling within the water tank 312. Optionally, the steam generator 300 may include a sterilizer and/or water filter (not shown) to purify the water and/or the steam. The steam generator 300 my further include a drain outlet (not shown) which is at or near the bottom of the water tank 312. Water may exit the steam generator 300 via a water outlet 328 connected to a waste pipeline. In one embodiment, a drain pump may forcibly drain the water out of the steam generator 300.

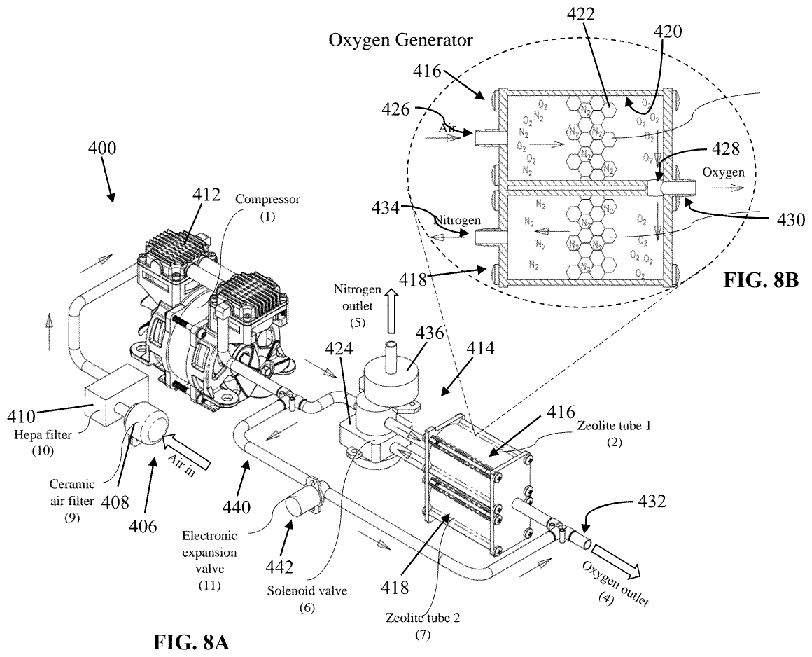

Referring to FIGS. 8A-8B, there is shown an exemplary embodiment of an oxygen generator 400 provided in accordance with aspects of the invention. The oxygen generator 400 may include a housing (not shown), an air inlet 406 with one or more air filters 408, 410 downstream of the air inlet 406. For example, in one embodiment, the oxygen generator 400 may include a first air filter 408 and a second air filter 410 for filtering out larger and smaller particles from the air, respectively. The first air filter 408 may be a ceramic air filter for removing relatively large particulate matter, e.g., larger than 5 microns. The second air filter 410 may be a high-efficiency particulate (HEPA) filter for removing relatively small particular matter, e.g., 0.3 microns. In one embodiment, the HEPA filter may be configured to remove approximately 99.97 percent of any dust, pollen, mold, bacterial, and other airborne particles. After being cleaned, the air may pass through a compressor 412 which pressurizes the clean air and forcibly feeds the air into an oxygen sieve or filter 414 for filtering out nitrogen such that the air stream contains a higher concentration of oxygen. The compressor 412 may comprise any desired commercially available compressor.

In one embodiment, the oxygen sieve 414 may comprise one or more zeolite filters 416, 418. In one embodiment, as shown in FIGS. 8A-8B, each zeolite filter 416, 418 may comprise a tube 420 with a plurality of zeolite pellets 422 therein. In one embodiment, the oxygen sieve 414 may utilize a pair of stacked upper and lower zeolite filters 416, 418 and a two-cycle filtration process, wherein each zeolite filter 416, 418 corresponds to a respective cycle and resulting flow path. For example, in one embodiment, a directional control valve 424, e.g., a solenoid valve, may selectively control the direction of airflow between a first cycle wherein air is directed into a first, upper zeolite filter 416 and a second cycle wherein the air is directed into a second, lower zeolite filter 418. In the first cycle, air may enter through a first air intake port 426 of the first zeolite filter 416, and the nitrogen particles may become trapped by the zeolite pellets 422, collecting at the front end of the first zeolite filter 416 (or left side thereof, as shown in FIG. 8B). Given the oxygen particles are smaller than the nitrogen particles, the oxygen particles will travel through the zeolite pellets 422 toward the rear end of the first zeolite filter 416 (or right side thereof, as shown in FIG. 8B). Post filtration, the oxygen particles may then travel out of the first zeolite filter 416 through a three-way or “T” connection port 428, which connects the first and second zeolite filters 416, 418 together with one another and further to a conjoined air outlet 430 of the oxygen sieve 414. Thereafter, the oxygen may travel out through an air outlet port 432 of the oxygen generator 400 and into the oxygen tube 402, and further into the facial dome 100 (FIG. 3). After a preset time period or an amount of nitrogen buildup in the first zeolite filter 416, the directional control valve 424 may reverse the flow path, initiating the second cycle. In the second cycle, the directional control valve 424 may direct air into a second air port 434 of the second, lower zeolite filter 418. Nitrogen particles may begin building up at the front end of the second filter 418, and oxygen particles may pass through the zeolite pellets 422 and thereafter exit through the connection port 428 and then through the air outlet port 430 of the oxygen sieve 414. Since the first and second zeolite filters 416, 418 are fluidly connected, a portion of the air may flow, in reverse, through the first zeolite filter 416, causing the previously captured nitrogen therein to exit the first zeolite filter 416. In particular, the nitrogen may exit through the first air port 426 and out through a nitrogen outlet 436 downstream of the directional control valve 424. Thereby, when cycling the first and second zeolite filters 416, 418, the reversed airflow will automatically cleanout the previously used zeolite filter 416, 418. For example, upon returning to the first cycle, a portion of the air may enter the second zeolite filter 418 and dispel the previously captured nitrogen out through the nitrogen outlet 436. The directional control valve 424 may reverse the airflow paths, cycling between the first and second cycles as desired, to filter oxygen and remove nitrogen from the zeolite filters 416, 418. The system controller 510 may control the directional control valve 424 based upon a predetermined time interval sensed by a filter timer and/or an oxygen sensor wherein flow reversal through the first and second zeolite filters 416, 418 is triggered upon detecting that an amount of oxygen has fallen below a predetermined, lower limit of oxygen (wherein a lower limit of oxygen in the airstream is indictive of an excessive buildup of nitrogen in a given zeolite filter 416, 418).

In one embodiment, the oxygen generator 400 may further include a regulator airstream pathway 440 for regulating the speed of the airstream provided by the oxygen generator 400. The regulator airstream pathway 440 may include an electronic expansion valve (EEV or EXV) 442 in parallel to the oxygen sieve 414 for adjusting the speed of the airstream outputted by the oxygen generator 400. In particular, after exiting the compressor 412, the airflow path may be split into a sieve airflow path (leading to the oxygen sieve 414) and a regulator flow path (leading to the regulator airstream pathway 440 that is parallel to the oxygen sieve 414). The airflow paths may rejoin one another downstream of the oxygen sieve 414 and upstream of the air outlet 432. Upon adjusting the electronic expansion valve 442, restricting or expanding the airflow therethrough, the airspeed at the air outlet 432 may be increased or decreased. The electronic expansion valve 442 may comprise any desired electronic expansion valve. Optionally, in one embodiment, the air outlet 432 may include a flow meter to sense the airflow speed therethrough. Therein, the system controller 510 may adjust the electronic expansion valve 442 as desired based on the sensed airflow speed from the flow meter and/or the oxygen sensor.

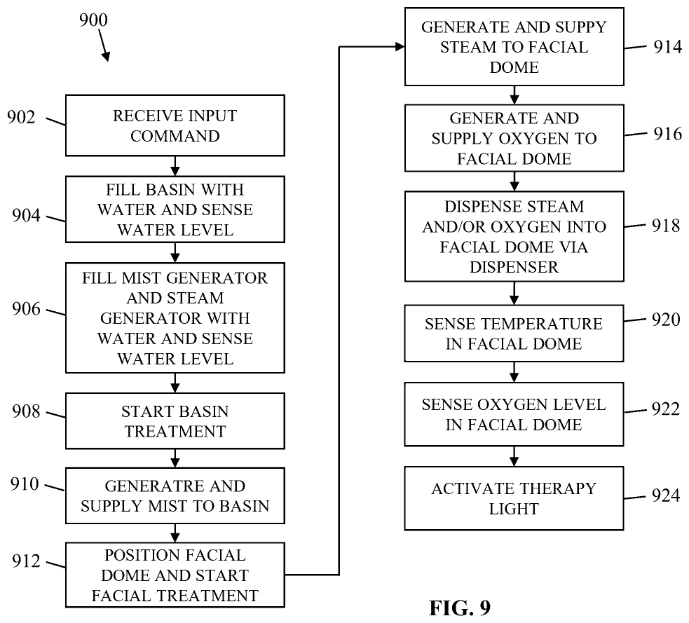

Referring to FIG. 9, there is shown a flowchart of a method 900 for providing one or more therapies or treatments to the user who is seated within the spa chair 10. Optionally, the spa chair 10 may sense whether a user is seated on the seat 14 via a pressure sensor. At step 902, the user seated in the spa chair 10 and/or the technician may select one or more therapies or treatments via the control panel 520. Responsive to the selected therapies or treatments, the system controller 510 may (de)activate the basin 18, the various generators 200, 300, 400, and/or the facial dome 100, simultaneously together or individually, as discussed in more detail herein. At step 904, the system controller 510 may fill the basin 18 with water and sense the water level therein via a basin water level sensor. At step 906, the system controller 510 may fill the water tanks 212, 312 of the mist generator 200 and the steam generator 300 with water and sense the water levels within the water tanks 212, 312. At step 908, the system controller 510 may start one or more basin treatments. For example, in one embodiment, the system controller 510 may selectively heat or cool the water within the basin 18 and utilize one or more jets to circulate the water within the basin 18.

At step 910, the system controller 510 may generate and supply mist to the basin 18 via the mist generator 200. The system controller 510 may automatically generate and supply mist to the basin 18 upon sensing a threshold water level in the basin 18 via the basin water level sensor and/or upon receiving a corresponding input command. Before activating the mist maker 216 of the mist generator 200, the system controller 510 may sense a water level within the water tank 212 of the mist generator 200 to ensure a sufficient amount of water is present within the water tank 212. The system controller 510 may activate the UV light 222 to sterilize the mist and/or water. The system controller 510 may also activate the fan 220 to forcibly blow mist out through the mist tube 202, through the mist dispenser spout 204, and into the basin 18. Thereafter, in one embodiment, the system controller 510 may sense the amount of mist within the basin 18 via the mist sensor and resupply mist based upon the sensed data from the mist sensor. The system controller 510 may also resupply mist based upon preset time intervals and/or an input command.

At step 912, the facial dome 100 may be positioned in its operational position, and the system controller 510 may start one or more facial therapies or treatments. As an initial step, the system controller 510 may sense whether the facial dome 100 is in its stowed or operational position via the dome position sensor, if equipped. Then, depending upon the user selection, the system controller 510 may automatically rotate the facial dome 100 from its stowed position into its operational position. For example, in one embodiment, if the user selects a facial therapy, the system controller 510 may initially sense that the facial dome 100 is in its stowed position and thereafter rotate the facial dome 100 into its operational position by activating the dome actuator 130. The system controller 510 may then sense the position of the facial dome 100 again to ensure that the facial dome 100 is positioned properly in the operational position. In one embodiment, the facial dome 100 may not comprise a dome actuator 130, and instead the facial dome 100 may be manually movable by the user or technician.

At step 914, the system controller 510 may generate and supply steam to the facial dome 100. The system controller 510 may activate the heating element 316 of the steam generator 300 which boils or nearly boils the water to create steam within the water tank 312. The steam may rise out of the water tank, as the steam is less dense than the surrounding air, and travel through the steam tube to the facial dome 100. At step 916, the system controller 510 may generate and supply oxygen to the facial dome 100. The system controller 510 may activate the compressor 412 and feed air through the oxygen sieve 414. The compressor 412 may drive the oxygen through the oxygen tube 402 and to the facial dome 100. At step 918, the dispenser 102 may dispense the oxygen and/or steam into the facial dome 100 via steam and oxygen outlets 154, 156 of the cover plate 142. In one embodiment, the system controller 510 may separately supply steam and oxygen to the facial dome 100. In one embodiment, the system controller 510 may simultaneously supply steam and oxygen to the facial dome 100.

Optionally, at step 920, the system controller 510 may sense the temperature within the facial dome 100 via the temperature sensor 106, if equipped. The system controller 510 may ensure that the ambient air within the facial dome 100 is maintained at a desired temperature or range thereof, and further that a maximum temperature limit is not exceeded. Thereby, the system controller 510 may deactivate the steam generator 300, thus terminating the stream of hot steam into the facial dome 100 and allowing the ambient air within the facial dome 100 to naturally cool off, and/or increase an amount of airflow from the oxygen generator 400 (increasing an amount of cooler air into the facial dome 100 by altering the speed of the compressor 412 and/or increasing the airflow through the electronic expansion valve 442). Optionally, at step 922, the system controller 510 may sense the oxygen level within the facial dome 100 via the oxygen sensor 106, if equipped. The system controller 510 may maintain a desired amount of oxygen within the facial dome 100 by activating and deactivating the oxygen generator 400 and/or throttling the electronic expansion valve 442, based upon the sensed oxygen level within the facial dome 100. In one embodiment, the facial dome 100 may not include a temperature sensor or an oxygen sensor, and instead the system controller 510 may intermittently (de)activate the steam generator 300 and/or the oxygen generator 400 based upon predetermined time intervals stored in the memory and/or an input command.

At step 924, the system controller 510 may activate the therapy light 104 of the facial dome 100 to perform one or more light therapies. The system controller 510 may activate the therapy light 104 alone or in combination with the steam and/or oxygen generators 300, 400. The system controller 510 may (de)activate the therapy light 104 based upon sensed light data from a light sensor, predetermined time intervals, and/or input commands. The therapy light 104 may provide visible light and/or invisible light therapies.

As can be appreciated, the system controller 510 may conduct multiple therapies or treatments simultaneously with one another. After completing the one or more therapies or treatments, the system controller 510 may initiate a cleaning cycle to clean the basin 18, the generators 200, 300, 400, and/or the facial dome 100. For example, in one embodiment, the system controller 510 may drain the basin 18 and apply a cleaning solution to thoroughly clean the basin 18 so that the basin 18 is ready for subsequent usage by another user. Also, for example, the system controller 510 may apply a cleaning solution to the mist generator 200, the steam generator 300, and/or the oxygen generator 400 to clean the generators for subsequent usage. Additionally, the system controller 510 may apply a cleaning solution to airstream within the mist tube 202, the steam tube 302 and/or the oxygen tube 402 to clean the dispenser and/or the facial dome 100. Furthermore, the system controller 510 may refill the basin 18 and/or the water tanks 212, 312 in preparation of any subsequent usage thereof.

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Each memory can have stored thereon instructions that when executed by a hardware processor cause the hardware processor to perform several tasks, including access data files, analyze data files, perform analysis of the data files, and provide outputs indicative of characteristics or parameters represented by the data files. Each hardware processor may comprise any desired processor.

Methods of making and of using the spa chair and components thereof, such as the facial dome and the one or more generators, are within the scope of the present invention.

Although limited embodiments of a pedicure chair or spa chair having a facial dome and one or more generators, and methods of operation thereof, have been specifically described and illustrated herein, many modifications and variations will be apparent to those skilled in the art. The method steps disclosed herein can be performed in a differing order as desired. The disclosure is also defined in the following claims.

### EXAMPLE EMBODIMENTS

The following are numbered example embodiments of the apparatuses, devices, systems, and methods related to spa chairs. The below listing of examples or any other examples disclosed herein may be combined in whole or in part. Elements of the examples disclosed herein are not limiting.

Example 1. A spa chair including a chair body, a seat having a seating surface, and a facial dome connected to the chair body. The facial dome is configured to direct steam and oxygen toward a head of a user. Optoinally, only one or the other stream of steam and oxygen is used or is operational at a time.

Example 2. The assembly, system, device, apparatus, and method of any of the above Examples alone or in combination, wherein the facial dome comprises a dispenser configured to dispense the steam and the oxygen toward the head of the user. Optionally, a static mixer may be used to facilitate mixing. Optionally each outlet for the steam and for the oxygen can include a manual directional distribution outlet.

Example 3. The assembly, system, device, apparatus, and method of any of the above Examples alone or in combination, wherein the dispenser is fitted within a dispenser cutout of a body of the facial dome.

Example 4. The assembly, system, device, apparatus, and method of any of the above Examples alone or in combination, wherein the dispenser cutout extends through the entire body of the facial dome from an outer surface to an inner surface thereof.

Example 5. The assembly, system, device, apparatus, and method of any of the above Examples alone or in combination, wherein the dispenser comprises an outer surface which is flush with an outer surface of the body of the facial dome when the dispenser is seated in the dispenser cutout and an inner surface opposite the outer surface. Optionally, the inner surface is directed toward the user and is flush with an inner surface of the body of the facial dome when the dispenser is seated in the dispenser cutout.

Example 6. The assembly, system, device, apparatus, and method of any of the above Examples alone or in combination, wherein the dispenser comprises a perimeter wall extending in between outer and inner surfaces. Optionally, the perimeter wall comprises integrated mating features configured to mate with complimentary mating features of the body of the facial dome when the dispenser is seated in the dispenser cutout.

Example 7. The assembly, system, device, apparatus, and method of any of the above Examples alone or in combination, wherein the dispenser comprises a housing including a base, a perimeter wall extending outwardly from the base, and an open top. The housing defines an tube compartment configured to receive and house a steam tube for transporting the steam and an oxygen tube for transporting the oxygen. Optionally, fewer tubes or additional tubes may be housed in the tube compartment.

Example 8. The assembly, system, device, apparatus, and method of any of the above Examples alone or in combination, wherein the dispenser comprises a cover plate connected to and covering the housing. The cover plate can include a steam outlet corresponding to the steam tube and an oxygen outlet corresponding to the oxygen tube.

Example 9. The assembly, system, device, apparatus, and method of any of the above Examples alone or in combination, wherein the base of the housing of the dispenser comprises a curvature which is complimentary to a curved outer surface of the facial dome such that an outer surface of the base is flush with the curved outer surface of the facial dome.

Example 10. The assembly, system, device, apparatus, and method of any of the above Examples alone or in combination, wherein the cover plate comprises a curvature which is complimentary to a curved inner surface of the facial dome such that an inner surface of the cover plate is flush with the inner surface of the facial dome. Optionally, the cover plate faces the head of the user when the cover plate is connected to the housing.

Example 11. The assembly, system, device, apparatus, and method of any of the above Examples alone or in combination, wherein the cover plate is transparent, and the cover plate is configured to cover a light source and further allow light emanating from the light source to pass therethrough such that the light shines onto the user through the cover plate.

Example 12. The assembly, system, device, apparatus, and method of any of the above Examples alone or in combination, wherein the spa chair further includes a therapy light connected to the facial dome and configured to administer a light therapy to the user.

Example 13. The assembly, system, device, apparatus, and method of any of the above Examples alone or in combination, wherein the spa chair further includes a therapy light housed within the dispenser.

Example 14. The assembly, system, device, apparatus, and method of any of the above Examples alone or in combination, wherein the therapy light comprises an array of lights on a light board.

Example 15. The assembly, system, device, apparatus, and method of any of the above Examples alone or in combination, wherein the therapy light comprise a plurality of LED lights on an LED light board.

Example 16. The assembly, system, device, apparatus, and method of any of the above Examples alone or in combination, wherein the therapy light comprises a left and a right bank of lights separated by a gap in between the right and right banks of lights.

Example 17. The assembly, system, device, apparatus, and method of any of the above Examples alone or in combination, wherein the gap in between the left and right banks of lights is configured to align with steam and oxygen tubes such that light emanating from the left and right banks is not obstructed by the steam and oxygen tubes.

Example 18. The assembly, system, device, apparatus, and method of any of the above Examples alone or in combination, wherein the therapy light emits light in the visible light spectrum and/or the invisible light spectrum, including ultraviolet light and infrared light.

Example 19. The assembly, system, device, apparatus, and method of any of the above Examples alone or in combination, wherein the therapy light is disposed within the housing of the dispenser and covered by the cover plate of the housing.

Example 20. The assembly, system, device, apparatus, and method of any of the above Examples alone or in combination, wherein the light board of the therapy light is complimentary in size and shape to the perimeter wall of the housing of the dispenser.

Example 21. The assembly, system, device, apparatus, and method of any of the above Examples alone or in combination, wherein the light board of the therapy light is press-fit into the housing of the dispenser.

Example 22. The assembly, system, device, apparatus, and method of any of the above Examples alone or in combination, wherein the therapy light is located next to the base of the housing of the dispenser, and the steam and oxygen tubes are positioned in between the therapy light and the cover plate of the dispenser.

Example 23. The assembly, system, device, apparatus, and method of any of the above Examples alone or in combination, wherein the cover plate of the dispenser comprises a steam outlet and an oxygen outlet that respectively receive the steam tube and the oxygen tube.

Example 24. The assembly, system, device, apparatus, and method of any of the above Examples alone or in combination, wherein the steam outlet and the oxygen outlet of the dispenser are vertically aligned with one another, which substantially aligns the steam and oxygen tubes, minimizing a profile of the steam and oxygen tubes such that the therapy light is unobstructed by the steam and oxygen tubes.

Example 25. The assembly, system, device, apparatus, and method of any of the above Examples alone or in combination, wherein the facial dome comprises a tube channel configured to receive and house a steam tube an oxygen tube therein.

Example 26. The assembly, system, device, apparatus, and method of any of the above Examples alone or in combination, wherein the tube channel comprises a steam tube channel and an oxygen tube channel for respectively housing the steam tube and the oxygen tube.

Example 27. The assembly, system, device, apparatus, and method of any of the above Examples alone or in combination, wherein the tube channel is located next to a bottom rim of the facial dome.

Example 28. The assembly, system, device, apparatus, and method of any of the above Examples alone or in combination, wherein the tube channel is internally disposed within the facial dome such that the steam tube and the oxygen tube are internally disposed and protected within the facial dome.

Example 29. The assembly, system, device, apparatus, and method of any of the above Examples alone or in combination, wherein the tube channel opens into the housing of the dispenser.

Example 30. The assembly, system, device, apparatus, and method of any of the above Examples alone or in combination, wherein the facial dome includes a pair of tube holes at its tapered ends for respectively receiving the steam tube and the oxygen tube therethrough.

Example 31. The assembly, system, device, apparatus, and method of any of the above Examples alone or in combination, wherein the facial dome is movable in between a retracted position when not in use and an extended, operational position wherein the facial dome substantially surrounds the head of the user.

Example 32. The assembly, system, device, apparatus, and method of any of the above Examples alone or in combination, wherein the spa chair further includes a steam generator configured to generate steam and provide the steam to the facial dome.

Example 33. The assembly, system, device, apparatus, and method of any of the above Examples alone or in combination, wherein the steam generator includes a water inlet, a water tank, a heating element disposed at a bottom of the water tank and configured to heat the water within the water tank, and a steam outlet fluidly connected to the steam tube disposed within the facial dome.

Example 34. The assembly, system, device, apparatus, and method of any of the above Examples alone or in combination, wherein the spa chair further includes an oxygen generator configured to generate oxygen and provide the oxygen to the facial dome.

Example 35. The assembly, system, device, apparatus, and method of any of the above Examples alone or in combination, wherein the oxygen generator includes an air inlet, a compressor, an oxygen sieve, and an oxygen outlet fluidly connected to the oxygen tube disposed within the facial dome.

Example 36. The assembly, system, device, apparatus, and method of any of the above Examples alone or in combination, wherein the oxygen generator further comprises one or more air filters configured for filtering the air upstream of the compressor.

Example 37. The assembly, system, device, apparatus, and method of any of the above Examples alone or in combination, wherein the oxygen sieve comprises one or more zeolite filters, each zoolite filter comprises a tube and a plurality of zeolite pellets disposed within the tube.

Example 38. The assembly, system, device, apparatus, and method of any of the above Examples alone or in combination, wherein the oxygen sieve comprises a pair of zeolite filters, including an upper and a lower zeolite filter configured to filter the airstream in a first and second cycle with opposite airflow path directions.

Example 39. The assembly, system, device, apparatus, and method of any of the above Examples alone or in combination, wherein the oxygen generator further comprises an airflow path solenoid valve configured to direct airflow into the upper zeolite filter in the first cycle and into the lower zeolite filter in the second cycle.

Example 40. The assembly, system, device, apparatus, and method of any of the above Examples alone or in combination, wherein the oxygen generator further comprises an electronic expansion valve in an auxiliary, regulator airflow path which is parallel to the airflow path of the oxygen sieve. The electronic expansion valve is configured to adjust a speed of the outputted airstream of the oxygen generator.

Example 41. The assembly, system, device, apparatus, and method of any of the above Examples alone or in combination, wherein the spa chair further comprises a system controller and a control panel. The system controller includes a processor and a memory.

Example 42. The assembly, system, device, apparatus, and method of any of the above Examples alone or in combination, wherein the system controller is operably connected to and configured to control the operation of the steam generator and the oxygen generator.

Example 43. The assembly, system, device, apparatus, and method of any of the above Examples alone or in combination, wherein the system controller is operably coupled to a temperature sensor located on or next to the facial dome. The temperature sensor is configured to sense a temperature of air within the facial dome and further detect a threshold temperature of the air within the facial dome. The system controller is configured to perform one or more of the following functions: to activate the steam generator and/or the oxygen generator upon an input command and to deactivate the steam generator upon receiving a threshold temperature signal from the temperature sensor when the temperature sensor detects the threshold temperature.

Example 44. The assembly, system, device, apparatus, and method of any of the above Examples alone or in combination, wherein the one or more dome sensors comprises a gas sensor configured to sense an amount of oxygen in the air within the facial dome. The system controller is configured to activate and deactivate the oxygen generator and/or adjust an airstream provided by the oxygen generator, based upon the sensed amount of oxygen from the oxygen sensor.

Example 45. The assembly, system, device, apparatus, and method of any of the above Examples alone or in combination, wherein the system controller is operably coupled to the facial dome, and the dispenser and therapy light thereof, and the system controller is further configured to control the output of the steam and oxygen and further control the operation of the therapy light.

Example 46. The assembly, system, device, apparatus, and method of any of the above Examples alone or in combination, wherein spa chair further comprises one or more dome sensors configured to sense data including a position of the dome, a characteristic of air within the facial dome, and/or a characteristic, trait, or status of the user within the facial dome.

Example 47. The assembly, system, device, apparatus, and method of any of the above Examples alone or in combination, wherein the one or more dome sensors include an optical sensor, a temperature sensor, a flowmeter sensor, a gas sensor, and/or a position sensor.

Example 48. The assembly, system, device, apparatus, and method of any of the above Examples alone or in combination, wherein the system controller is configured to initiate, terminate, and/or adjust the operation of the facial dome based upon the sensed data provided by the one or more dome sensors.

Example 49. The assembly, system, device, apparatus, and method of any of the above Examples alone or in combination, wherein the spa chair further comprises a temperature sensor located on or near the facial dome. The temperature sensor is configured to sense a threshold temperature and send a threshold temperature signal to the system controller. The system controller is configured to deactivate the steam generator and/or increase the airflow of the oxygen generator to cool the air within the facial dome upon receiving the threshold temperature signal.

Example 50. The assembly, system, device, apparatus, and method of any of the above Examples alone or in combination, wherein the spa chair further comprises a dome actuator operably connected to the system controller and connected to the facial dome, the dome actuator is configured to move the facial dome in between a stowed and an operational position.

Example 51. The assembly, system, device, apparatus, and method of any of the above Examples alone or in combination, wherein the dome actuator comprises a servo motor.

Example 52. The assembly, system, device, apparatus, and method of any of the above Examples alone or in combination, wherein the dome actuator is connected to and configured to rotate the facial dome cover.

Example 53. The assembly, system, device, apparatus, and method of any of the above Examples alone or in combination, wherein the spa chair further comprises a mist generator configured to generate and provide mist to a basin.

Example 54. The assembly, system, device, apparatus, and method of any of the above Examples alone or in combination, wherein the mist generator comprises a water inlet, a water tank, an ultrasonic mist maker, and a mist outlet fluidly coupled to a mist dispenser spout which is located adjacent to the basin.

Example 55. The assembly, system, device, apparatus, and method of any of the above Examples alone or in combination, wherein the system controller is operably coupled to the mist generator and is configured to automatically control the operation of the mist generator.

Example 56. The assembly, system, device, apparatus, and method of any of the above Examples alone or in combination, wherein mist generator further comprises a water level sensor within the water tank. The system controller is configured to fill or refill the water tank and further activate or deactivate the mist generator, based upon the sensed water level within the water tank.

Example 57. The assembly, system, device, apparatus, and method of any of the above Examples alone or in combination, wherein the system controller is operably connected to the steam generator, the oxygen generator, the therapy light, and the mist generator for providing a myriad of facial and pedicure therapies or treatments.

Example 58. The assembly, system, device, apparatus, and method of any of the above Examples alone or in combination, wherein the mist generator, the steam generator, and the oxygen generator are disposed and housed within the body of the spa chair.

Example 59. The assembly, system, device, apparatus, and method of any of the above Examples alone or in combination, wherein the spa chair includes one or more internal compartments for housing the mist generator, the steam generator, and the oxygen generator therein.

Example 60. A pedicure chair assembly comprising a chair body, a seat having a seating surface, and a basin located elevation-wise below the seating surface. The basin comprises walls and an open top. The basin defines an open cavity for holding water therein. The pedicure chair assembly further comprises a mist generator configured to generate and supply mist to the basin such that the mist obscures a view of the water within the basin.

Example 61. The assembly, system, device, apparatus, and method of any of the above Examples alone or in combination, wherein the mist generator is housed within the chair body.

Example 62. The assembly, system, device, apparatus, and method of any of the above Examples alone or in combination, wherein the pedicure chair assembly further comprises a mist dispenser spout located next to and elevation-wise above the basin, wherein the mist dispenser spout is fluidly coupled to the mist generator by a mist tube.

Example 63. The assembly, system, device, apparatus, and method of any of the above Examples alone or in combination, wherein the mist generator comprises, a water inlet, a water tank fluidly coupled to the water inlet and configured to house water therein, an ultrasonic mist maker disposed within the water tank and configured to generate mist from the water within the water tank, and a mist outlet fluidly coupled to the water tank.

Example 64. The assembly, system, device, apparatus, and method of any of the above Examples alone or in combination, wherein the mist generator further comprises an ultraviolet (UV) light disposed at a top of the water tank and configured to disinfect the water and/or mist.

Example 65. The assembly, system, device, apparatus, and method of any of the above Examples alone or in combination, wherein the mist generator further comprises a fan configured to blow mist out through the mist outlet and into the basin.

Example 66. A pedicure chair assembly comprising a chair body, a seat having a seating surface, and a basin located elevation-wise below the seating surface. The basin comprises walls and an open top. The basin defines an open cavity for holding water therein. The pedicure chair assembly further comprises a steam generator disposed within the chair body and configured to generate steam, an oxygen generator disposed within the chair body and configured to generate oxygen, and a mist generator disposed within the chair body and configured to generate and supply mist to the basin such that the mist obscures a view of the water within the basin. The pedicure chair assembly further comprises a facial dome connected to the chair body. The facial dome is fluidly connected to the steam generator and the steam generator. The facial dome is configured to respectively receive the steam and the oxygen from the steam generator and the oxygen generator, respectively, and direct the steam and the oxygen toward a head of a user.

What is claimed is:

1. A spa chair comprising:
a chair body;
a seat having a seating surface; and
a facial dome connected to the chair body, the facial dome configured to direct steam and oxygen toward a head of a user.

2. The spa chair of claim 1, wherein the facial dome comprises a dispenser configured to dispense the steam and the oxygen toward the head of the user.

3. The spa chair of claim 2, wherein the dispenser is fitted within a dispenser cutout of a body of the facial dome.

4. The spa chair of claim 3, wherein the dispenser comprises:
an outer surface which is flush with an outer surface of the body of the facial dome when the dispenser is seated in the dispenser cutout;
an inner surface opposite the outer surface, the inner surface is directed toward the user and is flush with an inner surface of the body of the facial dome when the dispenser is seated in the dispenser cutout; and
a perimeter wall extending in between the outer and inner surfaces, the perimeter wall comprising integrated mating features configured to mate with complimentary mating features of the body of the facial dome when the dispenser is seated in the dispenser cutout.

5. The spa chair of claim 2, wherein the dispenser comprises:
a housing comprising a base, a perimeter wall extending outwardly from the base, and an open top, the housing defining an tube compartment configured to receive and house a steam tube for transporting the steam and an oxygen tube for transporting the oxygen; and
a cover plate connected to and covering the housing, the cover plate comprising a steam outlet corresponding to the steam tube and an oxygen outlet corresponding to the oxygen tube.

6. The spa chair of claim 5, wherein the base of the housing of the dispenser comprises a curvature which is complimentary to a curved outer surface of the facial dome such that an outer surface of the base is flush with the curved outer surface of the facial dome.

7. The spa chair of claim 5, wherein the cover plate comprises a curvature which is complimentary to a curved inner surface of the facial dome such that an inner surface of the cover plate is flush with the inner surface of the facial dome, and the cover plate faces the head of the user when the cover plate is connected to the housing.

8. The spa chair of claim 5, wherein the cover plate is transparent, and the cover plate is configured to cover a light source and further allow light emanating from the light source to pass therethrough such that the light shines onto the user through the cover plate.

9. The spa chair of claim 1, further comprising a therapy light connected to the facial dome and configured to administer a light therapy to the user.

10. The spa chair of claim 2, further comprising a therapy light housed within the dispenser, wherein the therapy light comprises an array of lights on a light board.

11. The spa chair of claim 1, wherein the facial dome comprises a tube channel configured to receive and house a steam tube an oxygen tube therein.

12. The spa chair of claim 1, wherein the facial dome is movable in between a retracted position when not in use and an extended, operational position wherein the facial dome substantially surrounds the head of the user.

13. The spa chair of claim 1, further comprising:
a steam generator configured to generate steam and provide the steam to the facial dome; and
an oxygen generator configured to generate oxygen and provide the oxygen to the facial dome.

14. The spa chair of claim 13, further comprising:
a system controller operably coupled to the steam generator, the oxygen generator, and a temperature sensor located on or next to the facial dome, the temperature sensor configured to sense a temperature of air within the facial dome and further detect a threshold temperature of the air within the facial dome,
wherein the system controller is configured to activate the steam generator and/or the oxygen generator upon an input command, and
wherein the system controller is configured to deactivate the steam generator upon receiving a threshold temperature signal from the temperature sensor when the temperature sensor detects the threshold temperature.

15. A pedicure chair assembly comprising:
a chair body;
a seat having a seating surface;
a basin located elevation-wise below the seating surface, the basin comprising walls and an open top, the basin defining an open cavity for holding water therein; and
a mist generator configured to generate and supply mist to the basin such that the mist obscures a view of the water within the basin.

16. The pedicure chair assembly of claim 15, wherein the mist generator is housed within the chair body.

17. The pedicure chair assembly of claim 16, further comprising a mist dispenser spout located next to and elevation-wise above the basin, wherein the mist dispenser spout is fluidly coupled to the mist generator by a mist tube.

18. The pedicure chair assembly of claim 15, wherein the mist generator comprises:
a water inlet;
a water tank fluidly coupled to the water inlet and configured to house water therein;
an ultrasonic mist maker disposed within the water tank and configured to generate mist from the water within the water tank;
an ultraviolet (UV) light disposed at a top of the water tank and configured to disinfect the water and/or mist; and
a mist outlet fluidly coupled to the water tank.

19. The pedicure chair assembly of claim 18, wherein the mist generator further comprises a fan configured to blow mist out through the mist outlet and into the basin.

20. A pedicure chair assembly comprising:
a chair body;
a seat having a seating surface;
a basin located elevation-wise below the seating surface, the basin comprising walls and an open top, the basin defining an open cavity for holding water therein;
a steam generator disposed within the chair body and configured to generate steam;
an oxygen generator disposed within the chair body and configured to generate oxygen;
a mist generator disposed within the chair body and configured to generate and supply mist to the basin such that the mist obscures a view of the water within the basin; and
a facial dome connected to the chair body, the facial dome fluidly connected to the steam generator and the steam generator, and the facial dome configured to respectively receive the steam and the oxygen from the steam generator and the oxygen generator, respectively, and direct the steam and the oxygen toward a head of a user.
